(19) United States
(12) Patent Application Publication
(10) Pub. No.: US 2011/0034480 A1
(43) Pub. Date: Feb. 10, 2011

(54) COMPOSITIONS COMPRISING CETIRIZINE AND A NON BETA-2-ADRENORECEPTOR AGONIST, A BETA-2-ADRENORECEPTOR AGONIST OR AN ANTI-INFLAMMATORY AND THE USE THEREOF FOR THE TREATMENT OF RESPIRATORY DISORDERS

(76) Inventors: Malcolm Philip Young, Newcastle Upon Tyne (GB); Roy Drucker, Newcastle Upon Tyne (GB)

Correspondence Address: K&L Gates LLP, STATE STREET FINANCIAL CENTER, One Lincoln Street, BOSTON, MA, 02111-2950, US

(21) Appl. No.: 12/681,467

(22) PCT Filed: Oct. 3, 2008
(86) PCT No.: PCT/GB08/03338

§ 371 Date: Oct. 25, 2010

Related U.S. Application Data
(60) Provisional application No. 61/024,656, filed on Jan. 30, 2008.

(30) Foreign Application Priority Data
Oct. 5, 2007 (GB) 0719518.3

(51) Int. Cl. A61K 31/495; A61P 29/00; A61P 11/00

(52) U.S. Cl. 514/255.04

(57) ABSTRACT

There is described a pharmaceutical composition comprising a therapeutically effective amount of cetirizine, or a pharmaceutically acceptable derivative thereof, and a therapeutically effective amount of one or more compounds selected from the group consisting of a non beta-2-adrenoreceptor agonist; a beta-2-adrenoreceptor agonist and an anti-inflammatory agent; in admixture with a pharmaceutically acceptable adjuvant, diluent or carrier.

### FIELD OF THE INVENTION

The present invention provides medicaments and methods for the treatment of respiratory disorders.

More particularly the invention relates to the novel use of existing compounds for the treatment of respiratory disorders such as asthma, to methods of treatment related thereto and to novel pharmaceutical compositions.

### BACKGROUND

Asthma is a chronic disease of the respiratory system or airways in which the mucus around the airway constricts, becomes inflamed, and is lined with excessive amounts of mucus, often in response to one or more triggers. The trigger may be exposure to an environmental stimulant or allergen, cold air, exercise or exertion, or emotional stress. The airway constriction causes symptoms such as wheezing, shortness of breath, chest tightness, and coughing.

The symptoms of asthma, which can range from mild to life threatening, can usually be controlled with administration of a medicament, such as a bronchodilator, or a combination of medicaments.

In the UK alone 5.2 m people are currently receiving treatment for asthma, including 1.1 m children.

Treatment of asthma usually comprises the administration of relief medication and/or preventative medication. Relief medication is usually taken immediately to relieve asthma symptoms. Such medications quickly relax the muscles surrounding the narrowed airways, allowing the airways to open wider, making it easier to breathe again.

Relief medication usually comprises a short-acting, selective beta₂-adrenoceptor agonists, such as salbutamol, levalbuterol, terbutaline or bitolterol. Less selective relief medications include the use of adrenergic agonists, such as inhaled epinephrine and ephedrine tablets, or anticholinergic agents, such as ipratropium bromide.

Preventative medications include inhaled corticosteroids, which help to suppress inflammation and reduce the swelling of the lining of the airways. Preventive corticosteroids include ciclesonide, beclomethasone, budesonide, flunisolide, fluticasone, mometasone, and triamcinolone. However, long term use of corticosteroids is undesirable due to the existence of many side effects.

Antihistamines are commonly used to treat disorders like allergic rhinitis (also known as hay fever). Histamine is a chemical released from allergic cells in the body (such as mast cells) usually in response to an allergen. When histamine is released by allergic cells in the nose and eyes, the result is sneezing, runny nose, itchy eye/nose/throat, nasal congestion and post nasal-drip. Antihistamines are medications that block a receptor for histamine, thereby stopping the symptoms that histamine causes. Antihistamines are the most commonly used medications to treat allergic rhinitis. Examples of known antihistamines include diphenhydramine, chlorpheniramine and hydroxyzine, cetirizine, fexofenadine, desloratadine and loratidine.

It is also known to use treatments such as anti-histamines to treat the allergic symptoms that may underlie the chronic inflammation in asthma.

Thus, recently Wilson, Andrew M. “The Role of Antihistamines in Asthma Management”; Treatments in Respiratory Medicine, Volume 5, Number 3, 2006, pp. 149-158(10); described that antihistamines have been shown to have bronchodilatory effects, effects on allergen-, exercise-, and adenosine-monophosphate-challenge testing, and also to prevent allergen-induced nonspecific airways hyperresponsiveness. Moreover, Wilson reported that clinical studies have shown mixed results, and some studies have reported beneficial effects of azelastine, cetirizine, desloratadine, and fexofenadine on asthma symptoms or physiological measures in patients with asthma. Wilson also suggests that the combination of an antihistamine and a leukotriene receptor antagonist (such as montelukast) has been shown to have additive effects in certain studies.

One specific antihistamine is cetirizine. Cetirizine is 2-[2-[4-[(chlorphenyl)-phenyl-methyl]piperazin-1-yl]ethoxy]acetic acid and is described in European Patent Application No. 0 058 146.

Whilst Wilson reports that cetirizine may have beneficial effects on asthma symptoms, we have now found certain combination therapies comprising cetirizine in combination with certain other medicaments which is suitable for the treatment or alleviation of respiratory disorders.

Therefore, an objective of the present invention is to provide a novel and effective treatment for the treatment or alleviation of respiratory disorders.

### SUMMARY OF THE INVENTION

According to a first aspect of the invention we provide a pharmaceutical composition comprising a therapeutically effective amount of cetirizine, or a pharmaceutically acceptable derivative thereof, and a therapeutically effective amount of one or more compounds selected from the group consisting of a non beta-2-adrenoreceptor agonist; a beta-2-adrenoreceptor agonist and an anti-inflammatory agent; in admixture with a pharmaceutically acceptable adjuvant, diluent or carrier.

Pharmaceutically acceptable derivatives of the compounds of cetirizine include pharmaceutically acceptable salts, esters and amides of the carboxylic acid group. Suitable salts include ammonium, alkali metal (e.g. sodium, potassium and lithium) and alkaline earth metal (e.g. calcium or magnesium) salts, and salts with suitable organic bases, e.g. salts with hydroxylamine, lower alkylamines such as methylamine or ethylamine, with substituted lower alkylamines, e.g. hydroxy substituted alkylamines such as tris(hydroxymethyl)methylamine, or with simple monocyclic nitrogen heterocyclic compounds, e.g. piperidine or morpholine. Suitable esters include simple lower alkyl esters, e.g. the ethyl ester. The pharmaceutically acceptable acid addition salts include the hydrochloride, e.g. the dihydrochloride, the hydrobromide. The salts and esters may be made by conventional techniques, e.g. esterification or transesterification known per se.

Examples of non beta-2-adrenoreceptor agonists include pridefine, probenecid, nalidixic acid and acrosoxacin. Such compounds are known or may be prepared using conventional methods known per se.

Examples of beta-2-adrenoreceptor agonists include meluadrine and nardeterol. Such compounds are known or may be prepared using conventional methods known per se.

Examples of and anti-inflammatory agents include darbufelone, e.g. darbufelone mesilate, fluocinolone, e.g. fluocinolone acetonide, oxaprozin and mefenamic acid. Such compounds are known or may be prepared using conventional methods known per se.

In a preferred aspect of the invention a therapeutically effective amount of cetirizine, or a pharmaceutically acceptable derivative thereof, is combined with a therapeutically effective amount of an anti-inflammatory agent simultaneously, sequentially or separately. A preferred anti-inflammatory agent is mefenamic acid.

It is within the scope of the present invention for the composition to comprise more than one non beta-2-adrenoreceptor agonist; more than one beta-2-adrenoreceptor agonist or more than one anti-inflammatory agent. Furthermore, it is within the scope of the present invention for the composition to include cetirizine, or a pharmaceutically acceptable derivative thereof, in combination with two or more compounds selected from the group consisting of non beta-2-adrenoreceptor agonists; beta-2-adrenoreceptor agonists and anti-inflammatory agents hereinbefore described.

A particularly preferred composition of the present invention comprises a therapeutically effective amount of cetirizine, or a pharmaceutically acceptable derivative thereof, and a therapeutically effective amount of an anti-inflammatory agent; in admixture with a pharmaceutically acceptable adjuvant, diluent or carrier.

In this preferred aspect of the present invention the anti-inflammatory agent may be selected form one or more of darbufelone, e.g. darbufelone mesilate, fluocinolone, e.g. fluocinolone acetonide, oxaprozin and mefenamic acid. However, an especially preferred composition may comprise a therapeutically effective amount of cetirizine, or a pharmaceutically acceptable derivative thereof, and a therapeutically effective amount of mefenamic acid, or a pharmaceutically acceptable derivative thereof; in admixture with a pharmaceutically acceptable adjuvant, diluent or carrier.

The daily dose ratio of cetirizine to non beta-2-adrenoreceptor agonists; beta-2-adrenoreceptor agonists or anti-inflammatory agent may vary depending upon, inter alia, the specific nature of the combination therapy and/or the respiratory disorder to be treated. However, the ratio of cetirizine to non beta-2-adrenoreceptor agonist; beta-2-adrenoreceptor agonist or anti-inflammatory agent may be within the range 20,000, 000:1 to 1:250

The compositions of the invention may be administered in a daily dose regime. For example, a suitable daily dosage of cetirizine hydrochloride may be 10 mg to be taken at bedtime for up to seven days, whilst a suitable daily dosage for non beta-2-adrenoreceptor agonist; beta-2-adrenoreceptor agonist or anti-inflammatory agent may be, for example as mefenamic acid, 1000 mg to be taken as 250 mg four times daily, for up to seven days.

Therefore, in accordance with an additional aspect, the present invention provides the use of cetirizine, or a pharmaceutically acceptable derivative thereof, and one or more of a non beta-2-adrenoreceptor agonists; a beta-2-adrenoreceptor agonists and an anti-inflammatory agents; in the manufacture of a medicament for the treatment or alleviation of a respiratory disorder.

In addition, the present invention provides cetirizine, or a pharmaceutically acceptable derivative thereof, and one or more of a non beta-2-adrenoreceptor agonists; a beta-2-adrenoreceptor agonists and an anti-inflammatory agents; as a combination therapy for administration simultaneously, sequentially or separately, for the treatment or alleviation of a respiratory disorder.

The present invention further provides mefenamic acid as a combination therapy for administration simultaneously, sequentially or separately, with cetirizine, or a pharmaceutically acceptable derivative thereof, for the treatment or alleviation of a respiratory disorder. The invention also provides the use of mefenamic acid in the manufacture of a medicament for the treatment or alleviation of a respiratory disorder and particularly in the manufacture of a combination therapy with cetirizine, or a pharmaceutically acceptable derivative thereof, for administration simultaneously, sequentially or separately.

More specifically, the present invention provides the use of cetirizine or a pharmaceutically acceptable derivative thereof, in the manufacture of a combination therapy for the treatment or alleviation of a respiratory disorder. Said combination therapy particularly comprising cetirizine and one or more of a non-beta-2-adrenoreceptor agonists; a beta-2-adrenoreceptor agonists and an anti-inflammatory agents as hereinbefore described.

The use according to the invention may comprise the manufacture of a medicament as hereinbefore described for administration simultaneously, sequentially or separately. Optionally, the use comprises the manufacture of a medicament for simultaneous administration and therefore, the use comprises the manufacture of a composition as hereinbefore described.

Furthermore, in a further aspect, the present invention provides a method of treatment of a patient suffering from a respiratory disorder, said method comprising the administration of a therapeutically effective amount of a combination therapy comprising cetirizine or a pharmaceutically acceptable derivative thereof, and one or more of a non beta-2-adrenoreceptor agonists; a beta-2-adrenoreceptor agonists and an anti-inflammatory agents.

The method of the invention may comprise the administration of a combination therapy comprising cetirizine, or a pharmaceutically acceptable derivative thereof, and one or more of a non beta-2-adrenoreceptor agonists; a beta-2-adrenoreceptor agonists and an anti-inflammatory agents; simultaneously, sequentially or separately.

Preferably, the method of the invention comprises the simultaneous administration of cetirizine or a pharmaceutically acceptable derivative thereof, and one or more of a non beta-2-adrenoreceptor agonists; a beta-2-adrenoreceptor agonists and an anti-inflammatory agents. Thus, with such simultaneous administration the cetirizine, or a pharmaceutically acceptable derivative thereof, and one or more of a non beta-2-adrenoreceptor agonists; a beta-2-adrenoreceptor agonists and an anti-inflammatory agents; may be made up as a composition according to the invention as hereinbefore described. However, it will be understood by the person skilled in the art that the method of the invention may comprise the separate or sequential administration of cetirizine or a pharmaceutically acceptable derivative thereof, and one or more of a non beta-2-adrenoreceptor agonists; a beta-2-adrenoreceptor agonists and an anti-inflammatory agent.

In the use or method of the invention as hereinbefore described, when the cetirizine, or a pharmaceutically acceptable derivative thereof, and one or more of a non beta-2-adrenoreceptor agonists; a beta-2-adrenoreceptor agonists and an anti-inflammatory agents; are administered separately, it is within the scope of the invention for the active agents at different times and in different dosage regimes. Thus, for example, cetirizine may be administered as a once daily dose whilst the one or more of a non beta-2-adrenoreceptor agonists; a beta-2-adrenoreceptor agonists and an anti-inflammatory agents; may be administered form 2 to 6 times daily, such as 4 times daily.

Advantageously, the composition of the invention may be administered orally.

Compositions of the invention may be administered in combination with one or more other treatments known per se. Examples of other medicaments known to be efficacious in the treatment or alleviation of a respiratory disorder include, but shall not be limited to, β2-agonists, e.g. fenoterol, formoterol, pirbuterol, reproterol, rimiterol, salbutamol, salmeterol and terbutaline; non-selective beta-stimulants such as isoprenaline; xanthine bronchodilators, e.g. theophylline, aminophylline and choline theophyllinate; anticholinergics, e.g. ipratropium bromide; mast cell stabilisers, e.g. sodium chromoglycate and ketotifen; bronchial anti-inflammatory agents, e.g. nedocromil sodium; and steroids, e.g. beclomethasone dipropionate, fluticasone, budesonide, flunisolide and ciclesonide, and isomers and/or salts or derivatives thereof. In addition, other combination therapies which may be co-administered with the composition of the present invention include, but shall not be limited to, combinations of steroids, such as, beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; and flunisolide and salmeterol.

Thus, in the use, method and/or composition of the invention each of the composition of the invention may be put up as a tablet, capsule, dragee, suppository, suspension, solution, injection, e.g. intravenously, intramuscularly or intraperitoneally, implant, a topical, e.g. transdermal, preparation such as a gel, cream, ointment, aerosol or a polymer system, or an inhalation form, e.g. an aerosol or a powder formulation. Preferably, the composition of the invention may be administered enterally, e.g. orally.

Compositions suitable for enteral/oral administration include tablets, capsules, dragees, liquid suspensions, solutions and syrups;

compositions suitable for topical administration to the skin include creams, e.g. oil-in-water emulsions, water-in-oil emulsions, ointments or gels;
examples of such adjuvants, diluents or carriers are:
for tablets and dragees—fillers, e.g. lactose, starch, microcrystalline cellulose, talc and stearic acid; lubricants/glidants, e.g. magnesium stearate and colloidal silicon dioxide; disintegrants, e.g. sodium starch glycolate and sodium carboxymethylcellulose;
for capsules—pregelatinised starch or lactose;
for oral or injectable solutions or enemas—water, glycols, alcohols, glycerine, vegetable oils;
for suppositories—natural or hardened oils or waxes.

In a further embodiment, the compositions and methods described herein may be used prophylactically as a means to prevent the development and/or onset of respiratory disorder.

The term respiratory disorder will be understood by the person skilled in the art to include, inter alia, asthma, allergic asthma, so-called ‘intrinsic’ asthma (in which no sensitivity to extrinsic antigen can be demonstrated), reversible obstructive airways disease (ROAD), chronic obstructive pulmonary disorder (COPD), bronchitis, respiratory infections, coughs and the bronchial obstruction associated with the common cold.

The invention will now be illustrated by way of example only.

### DETAILED DESCRIPTION

### Example 1

### Phase IIa Clinical Study

In a Phase II clinical study designed to evaluate efficacy in a small patient population. 40 patients were included in a trial:

20 were administered their existing inhaled medication

20 were administered their existing inhaled medication on existing plus therapy comprising cetirizine hydrochloride and mefenamic acid.

cetirizine hydrochloride (10 mg) was administered orally as a bedtime dose
mefenamic acid (250 mg) was administered orally four times daily.

### Results

A diminution of recourse to inhalers was observed:

Over the period of the course, 16% of those on existing medication who needed their inhalers in week 1 were independent of their inhalers in week 12.42% of those taking the cetirizine hydrochloride/mefenamic acid therapy that needed their inhalers in week 1 were independent of their inhalers in week 12.

40% of those on existing medication did not need their inhalers in week 12, but 62% of those on the cetirizine hydrochloride/mefenamic acid therapy did not need, their inhalers in week 12.

Patients self reports on their condition:

Report scores decreased in the cetirizine hydrochloride/mefenamic acid therapy patients by a factor of 43%, which was statistically significant (P<0.05), even in so small a sample.

1. A pharmaceutical composition comprising a therapeutically effective amount of cetirizine, or a pharmaceutically acceptable derivative thereof, and a therapeutically effective amount of one or more compounds selected from the group consisting of a non beta-2-adrenoreceptor agonist; a beta-2-adrenoreceptor agonist and an anti-inflammatory agent; in admixture with a pharmaceutically acceptable adjuvant, diluent or carrier.

2. A pharmaceutical composition according to claim 1 wherein the pharmaceutically acceptable derivative of cetirizine is a salt.

3. A pharmaceutical composition according to claim 1 wherein the pharmaceutically acceptable derivative of cetirizine is the hydrochloride salt.

4. (canceled)

5. (canceled)

6. A pharmaceutical composition according to claim 1 wherein the composition comprises a therapeutically effective amount of cetirizine, or a pharmaceutically acceptable derivative thereof, and a therapeutically effective amount of an anti-inflammatory agent.

7. (canceled)

8. A pharmaceutical composition according to claim 1 wherein the anti-inflammatory agent is mefenamic acid, or a derivative thereof.

9. (canceled)

10. A pharmaceutical composition according to claim 1 wherein the composition is suitable for oral administration.

11. (canceled)

12. A pharmaceutical composition according to claim 1 wherein the ratio of cetirizine, or a derivative thereof, to non beta-2-adrenoreceptor agonists; beta-2-adrenoreceptor agonists or anti-inflammatory agent is within the range 20,000, 000:1 to 1:250.

13. A pharmaceutical composition according to claim 1 wherein the daily dosage of cetirizine hydrochloride is from 5 mg to 20 mg.

14. (canceled)

15. A pharmaceutical composition according to claim 1 wherein the daily dosage non beta-2-adrenoreceptor agonist; beta-2-adrenoreceptor agonist or anti-inflammatory agent is from 1 microgram to 500 mg.

16. (canceled)

17. A pharmaceutical composition according to claim 1 wherein the daily dosage of cetirizine hydrochloride is 10 mg and the daily dosage of mefenamic acid is 1000 mg.

18-39. (canceled)

40. A method of treatment of a patient suffering from a respiratory disorder, said method comprising the administration of a therapeutically effective amount of a combination therapy comprising cetirizine, or a pharmaceutically acceptable derivative thereof, and one or more compounds selected from the group comprising a non beta-2-adrenoreceptor agonist; a beta-2-adrenoreceptor agonist and an anti-inflammatory agent.

41. A method according to claim 40 wherein the method comprises the administration of cetirizine, or a pharmaceutically acceptable derivative thereof, and one or more compounds selected from the group comprising a non beta-2-adrenoreceptor agonist; a beta-2-adrenoreceptor agonist and an anti-inflammatory agent; simultaneously, sequentially or separately.

42. A method according to claim 40 wherein the method comprises the simultaneous administration of cetirizine, or a pharmaceutically acceptable derivative thereof, and one or more compounds selected from the group comprising a non beta-2-adrenoreceptor agonist; a beta-2-adrenoreceptor agonist and an anti-inflammatory agent.

43. (canceled)

44. The method according to claim 40 wherein the pharmaceutically acceptable derivative of cetirizine is a salt.

45. The method according to claim 40 wherein the pharmaceutically acceptable derivative of cetirizine is the hydrochloride salt.

46-48. (canceled)

49. The method according to claim 40 wherein the use comprises the manufacture of a medicament comprising cetirizine, or a pharmaceutically acceptable derivative thereof, and an anti-inflammatory agent.

50. The method according to claim 40 wherein the anti-inflammatory agent is mefenamic acid, or a derivative thereof.

51. (canceled)

52. The method according to claim 40 wherein the method comprises oral administration.

53. (canceled)

54. The method according to claim 40 wherein the ratio of cetirizine, or a derivative thereof, to non beta-2-adrenoreceptor agonists; beta-2-adrenoreceptor agonists or anti-inflammatory agent is within the range 20,000, 000:1 to 1:250.

55. The method according to claim 40 wherein the daily dosage of cetirizine hydrochloride is from 5 mg to 20 mg.

56. (canceled)

57. The method according to claim 40 wherein the daily dosage non beta-2-adrenoreceptor agonist; beta-2-adrenoreceptor agonist or anti-inflammatory agent is from 1 microgram to 500 mg.

58. (canceled)

59. The method according to claim 40 wherein the daily dosage of cetirizine hydrochloride is 10 mg and the daily dosage of mefenamic acid is 1000 mg.

60. The method according to claim 40 wherein the medicaments are administrable on a once or a twice daily dose regime.

61. Cetirizine, or a pharmaceutically acceptable derivative thereof, and one or more of a non beta-2-adrenoreceptor agonists; a beta-2-adrenoreceptor agonists and an anti-inflammatory agents; as a combination therapy for administration simultaneously, sequentially or separately, for the treatment or alleviation of a respiratory disorder.

62. Mefenamic acid as a combination therapy for administration simultaneously, sequentially or separately, with cetirizine, or a pharmaceutically acceptable derivative thereof, for the treatment or alleviation of a respiratory disorder.

63. (canceled)
